(19) United States
(12) Patent Application Publication
(10) Pub. No.: US 2022/0135580 A1
(43) Pub. Date: May 5, 2022

(54) HYDROXYPHEOPHORBIDE COMPOUNDS, METHODS AND USES THEREOF

(71) Applicants: CIIMAR - CENTRO INTERDISCIPLINAR DE INVESTIGAÇÄO MARINHA E AMBIENTAL, MATOSINHOS (PT); UNIVERSIDADE DO PORTO, Porto (PT)

(72) Inventors: Ralph URBATZKA, MATOSINHOS (PT); Sara FREITAS, MATOSINHOS (PT); Pedro Nuno COSTA LEÃO, MATOSINHOS (PT); Maria Ligia SOUSA, MATOSINHOS (PT); Mariana ALVES REIS, MATOSINHOS (PT); Natália GONÇALVES SILVA, MATOSINHOS (PT); Vitor Manuel DE OLIVEIRA E VASCONCELOS, MATOSINHOS (PT); Tiago RIBEIRO, MATOSINHOS (PT); Filipa ROSA, MATOSINHOS (PT)

(21) Appl. No.: 17/435,663

(22) PCT Filed: Mar. 2, 2020
(86) PCT No.: PCT/IB2020/051759

§ 371 Date: Sep. 1, 2021

(30) Foreign Application Priority Data
Mar. 1, 2019 (IT) 115348

(51) Int. Cl. C07D 487/22; A61P 3/04; A23L 33/10

(52) U.S. Cl. CPC C07D 487/22; A23L 33/10; A61P 3/04

(57) ABSTRACT

The present disclosure relates to hydroxypheophorbide compounds with bioactivities towards obesity and obesity-related co-morbidities. Therefore, the present subject-matter discloses hydroxypheophorbide compounds or a pharmaceutically acceptable salt, hydrate, solvate, N-oxide, stereoisomer, diastereoisomer, enantiomer or atropisomer, polymorph for use in medicine comprising formula (I), wherein R is CₙH₍₂ₙ₋₁₎ and n is an entire number multiple of 5. The compound of the present disclosure may be use in the therapy or treatment of obesity, an obesity-related disorder, an obesity related disease, being overweight, an obesity-related condition, or lipid obesity disorders.

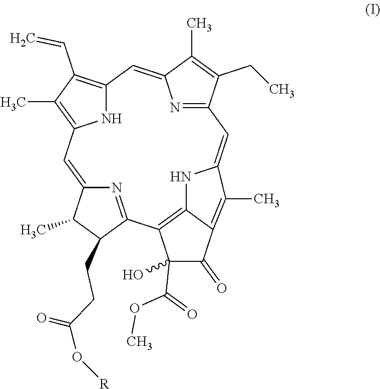

### TECHNICAL FIELD

The present disclosure relates to 13²-hydroxypheophorbide compounds with bioactivities towards obesity or obesity-related co-morbidities.

### BACKGROUND

Obesity is an increasing epidemic, since a higher percentage of the world's population is overweight, and is associated with several chronic diseases like diabetes, cardiovascular diseases and cancer. Some natural products are being used in the clinic as anti-obesogenic compounds, as for example Orlistat, a synthetic derivative of lipostatin isolated from Streptomyces toxytricini.

Chlorophyll molecules and their main derivatives (pheophytins, pheophorbides) are known for a long time. Some beneficial bioactivities were described for them including (1) antioxidant properties (Kang et al., 2018), (2) anti-inflammatory activity (Kang et al., 2018; Subramoniam et al., 2012), (3) anti-mutagenic activity (Ferruzzi et al., 2002), (4) cytotoxic effects on cancer cells (Zhao et al., 2014), (5) antiviral activity against hepatitis C virus (Wang et al., 2009), (6) antimicrobial activity (Gomes et al., 2015) and (7) induction of neuro-differentiation (Ina et al., 2007). Reduction of lipid content in murine 3T3L1 cells was shown for a pheophytin rich extract of a plant at 100 μg/ml (Seemann et al., 2018). However, for 13²-(S)-hydroxy-pheophytin a (hpa) only few bioactivities are known. Hpa was shown to have a low cytotoxicity on cancer cells (8.8-22 μg/ml, Cheng et al., 2001,). A methanolic extract from red algae containing hpa amongst 7 other compounds (major peaks by unidentified compounds) showed anti-inflammatory activity in vitro at 10 μg/ml (Shu et al., 2013). Until this moment, no utility was demonstrated for hpa in relation to obesity, or obesity-related co-morbidities as diabetes or fatty liver disease or any effects of lipid metabolic diseases.

These facts are disclosed in order to illustrate the technical problem addressed by the present disclosure.

### General Description

The hydroxypheophorbide compounds can be used for reduction of lipids, and possibly for the treatment of obesity or related diseases. They have the advantage that they are from a natural resource and can be marketed as natural products.

In the present disclosure a known and an unprecedented hydroxypheophorbide were isolated from marine cyanobacteria: 13²-(S)-hydroxy-pheophytin a (hpa), and the 13²-(S)-hydroxy-pheofarnesin a (hfa), respectively. Hpa and hfa demonstrated significant lipid reducing activities with an IC₅₀ of 8.9±0.4 and 15.5±1.3 μM, respectively, in the zebrafish Nile red fat metabolism assay. Bioactivity was further confirmed in differentiated murine pre-adipocytes grown as 3D spheroids. Since these molecules are shown to be produced from organisms that satisfy the GRAS criteria (generally regarded as safe), their lipid reducing activities can be marketed as novel natural products in the form of nutraceuticals or food supplements for the treatment of obesity or related co-morbidities. Commercialization as isolated molecules or as raw material would be also possible.

The hdroxypheophorbide compounds of the presented disclosure were isolated by bio-assay guided fractionation approaches based on the zebrafish Nile red fat metabolism assay. 13²-(S)-Hydroxy-pheofarnesin a (hfa) is a novel compound, reported for the first time. Moreover, it is disclosed for the first time that hpa and hfa have lipid reducing activities in two different model systems, in the zebrafish Nile red fat metabolism assay and in differentiated murine pre-adipocytes grown as 3D spheroids

An aspect of the present disclosure relates to hydroxypheophorbide compounds with bioactivities towards obesity and obesity-related co-morbidities.

Therefore, the present subject-matter discloses compounds or a pharmaceutically acceptable salt, hydrate, solvate, N-oxide, stereoisomer, diastereoisomer, enantiomer or atropisomer, polymorph for use in medicine comprising the following formula

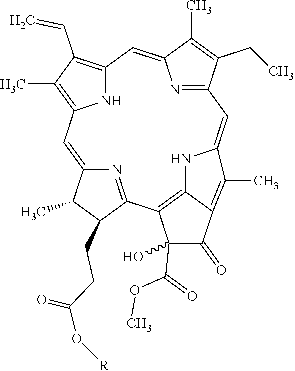

wherein R is CₙH₍₂ₙ₋₁₎ and n is an entire number multiple of 5.

In an embodiment, n may be selected from 5 or 10 or 15 or 20 or 25 or 30, preferably 15 or 20.

In an embodiment, R may be C₅H₉, C₁₀H₁₉ or C₁₅H₂₉ or C₂₀H₃₉ or C₂₅H₄₉ or C₃₀H₅₉, preferably C₁₅H₂₉ or C₂₀H₃₉.

In an embodiment, R may be

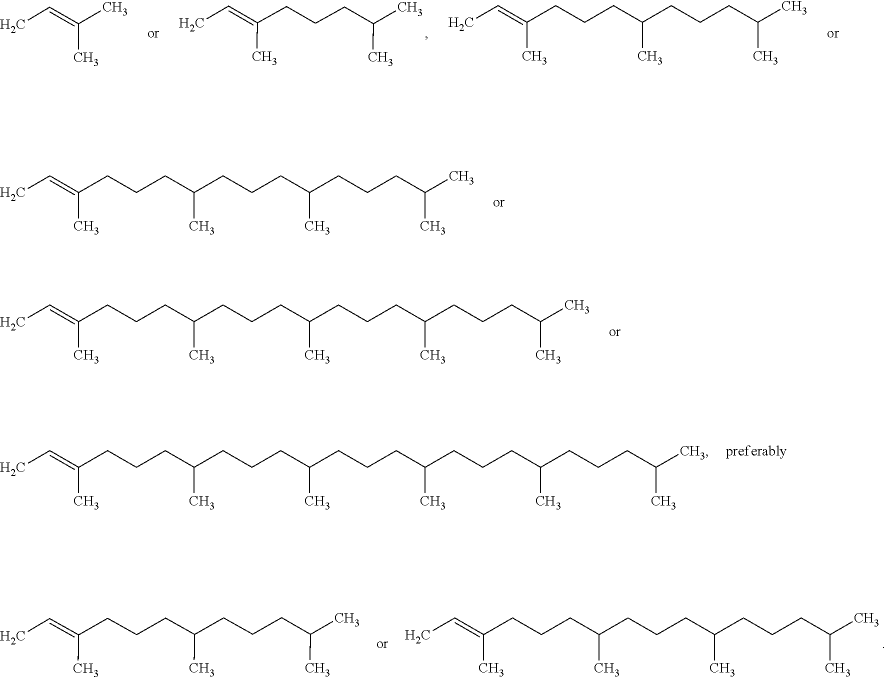

In an embodiment, the compound of the present disclosure may be use in the therapy or treatment of metabolic diseases. Preferably in therapy or treatment of obesity, an obesity-related disorder, an obesity related disease, being overweight, an obesity-related condition, or lipid obesity disorders. More preferably, morbid obesity, visceral fat obesity, diabetes, insulin-resistance syndrome, nonalcoholic steatohepatitis, a cardiovascular disorder, renal disorders, pancreatic disorders, polycystic ovary syndrome, metabolic syndrome or fatty liver.

In an embodiment for better results, the compound is:

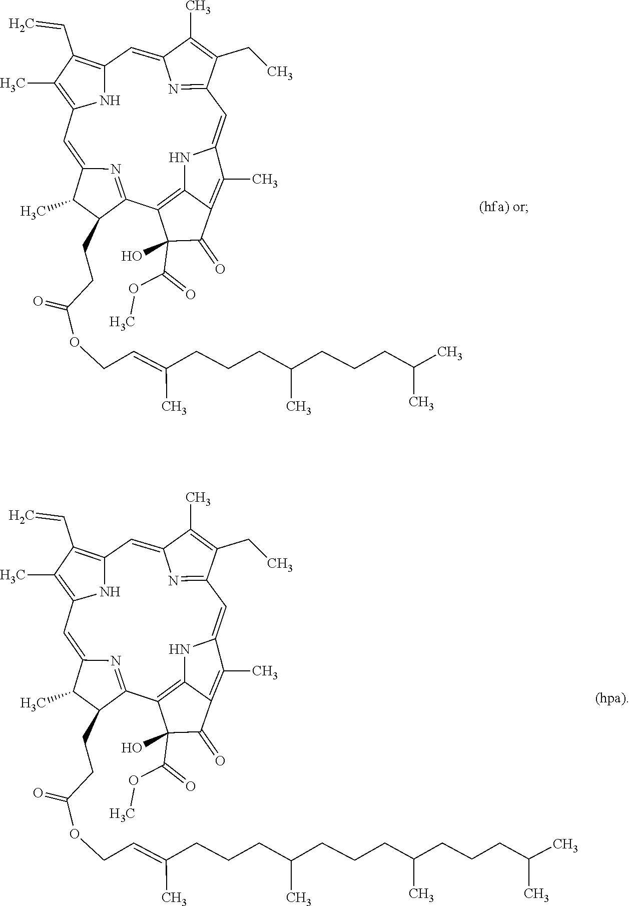

Another aspect of the present disclosure is a composition comprising at least a compound, pharmaceutically acceptable salt, hydrate, solvate, N-oxide, stereoisomer, diastereoisomer, enantiomer, atropisomer, or polymorph thereof comprising the following formula:

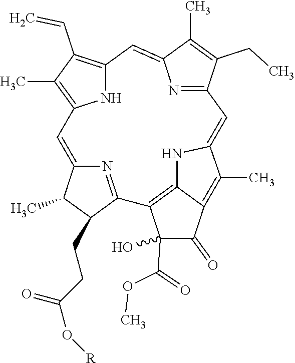

wherein R is CₙH₍₂ₙ₋₁₎

n is an entire number multiple of 5 and

n is different from 20. Preferably, wherein n is selected from 5 or 10 or 15 or 25 or

30, more preferably n is 15.

In an embodiment, R may be C₅H₉, C₁₀H₁₉ or C₁₅H₂₉ or C₂₅H₄₉ or C₃₀H₅₉, preferably C₁₅H₂₉

In an embodiment, R may be

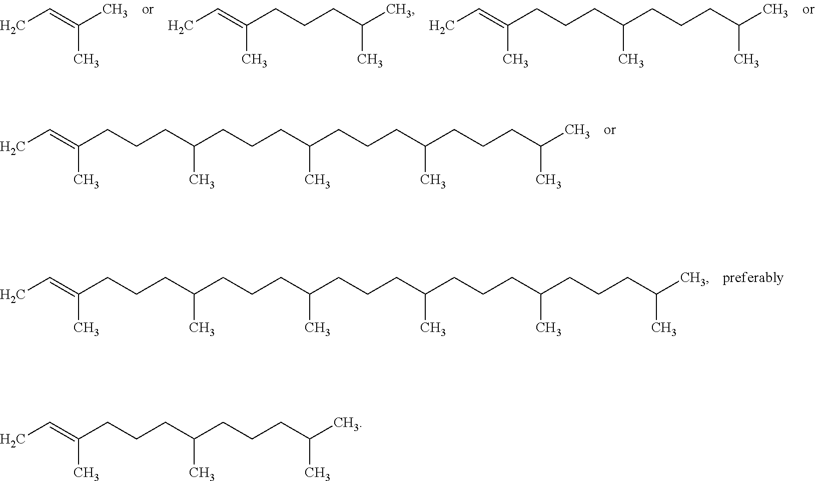

Another aspect of the present disclosure relates to a composition comprising at least a compound, pharmaceutically acceptable salt, hydrate, solvate, N-oxide, stereoisomer, diastereoisomer, enantiomer, atropisomer, or polymorph thereof, as defined in any of the previous claims and a pharmaceutically acceptable carrier, adjuvant, excipient, emulsification agent, or mixtures thereof.

In an embodiment, the composition is a pharmaceutical composition, a foodstuff composition, a dietary composition, a nutraceutical composition or an anti-obesity composition.

Another aspect of the present disclosure relates to a foodstuff or a nutraceutical comprising the compound or composition described in the present disclosure.

Another aspect of the present disclosure relates to the use of a hydroxypheophorbide compound or composition as an anti-obesity agent.

Another aspect of the present disclosure relates to a method of treating or preventing obesity or related diseases comprising administering a therapeutically effective amount of a compound or a composition as defined in any of the previous claims.

Another aspect of the present disclosure relates to a process for the production of the compound of formula

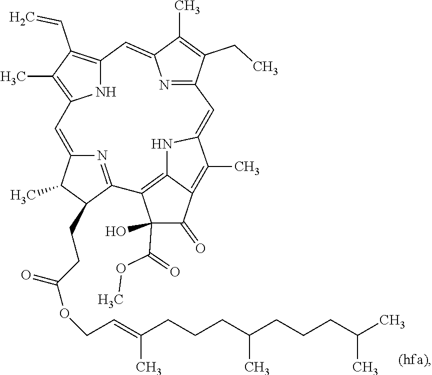 [SMILES: C=Cc1c(C)c2cc3nc(c4c5[nH]c(cc6nc(cc1[nH]2)C(C)=C6CC)c(C)c5C(=O)[C@]4(O)C(=O)OC)[C@@H](CCC(=O)OC/C=C(\C)CCCC(C)CCCC(C)C)[C@@H]3C]

and its isolation from an extract of Nodosilinea sp. LEGE06001.

Another aspect of the present disclosure relates to the compound comprises the formula

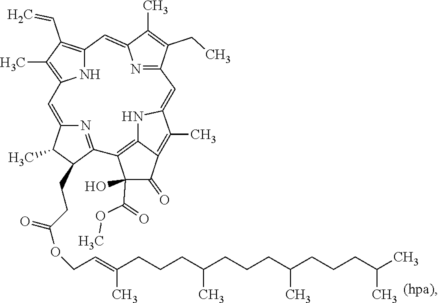 [SMILES: C=Cc1c(C)c2cc3nc(c4c5[nH]c(cc6nc(cc1[nH]2)C(C)=C6CC)c(C)c5C(=O)[C@]4(O)C(=O)OC)[C@@H](CCC(=O)OC/C=C(\C)CCCC(C)CCCC(C)CCCC(C)C)[C@@H]3C]

and its isolation from an extract of Cyanobium sp. LEGE 07175.

### BRIEF DESCRIPTION OF THE DRAWINGS

The following figures provide preferred embodiments for illustrating the disclosure and should not be seen as limiting the scope of invention.

FIG. 1.A: Representative images of the zebrafish Nile red fat metabolism assay. Nile red fluorescence signal indicates neutral lipids around the yolk sac and stomach/intestine. Compound 1 (hpa) and compound 2 (hfa) show a decrease in the Nile red staining that is not present when zebrafish were exposed to chlorophylls a and b. A solvent control (0.1% dimethyl sulfoxide, DMSO) and positive control group (50 μM resveratrol, REV) was included.

FIG. 1.B: Quantification of lipid-reducing activity in the zebrafish Nile Red fat metabolism assay after exposure for 48 h. Values are expressed as mean fluorescence intensity (MFI) relative to the DMSO group and are derived from 6 to 8 individual larvae per treatment group. The data are represented as box-whisker plots (5 to 95 percentiles). Asterisks highlight significant differences compared to the DMSO group (****p<0.0001; ***p<0.001; **p<0.01; *p<0.05).

FIG. 2: Evaluation of lipid content (Nile red) and viability (calcein AM) in differentiated 3T3-L1 spheroids after exposure to compound 1 (hpa) and compound 2 (hfa) over 48 h. Panel A shows the results of quantification of fluorescence by CellProfiler software, and Panel B some representative images from fluorescence microscopy (both fluorescence channels were converted into black and white for better visualization). Statistical difference to the solvent control were analysed by One-Way ANOVA, followed by a Dunnett's multiple comparison post-test (***p-value<0.001, **p-value <0.01, *p-value <0.05). Quantification of free glycerol (lipolysis) on the medium where 3T3-L1 organoids were exposed to compound 1 (hpa) and compound 2 (hfa) over 48 h. No significant alterations on free glycerol content in the medium were observed. Kolmogorov-Smirnov test was used to test normality of the data, followed by a Dunnett's multiple comparison post-test (***p-value <0.001, **p-value <0.01, *p-value <0.05).

FIG. 3: Comparative quantification of hpa from different sources. A Total Ion Chromatogram (TIC) from hpa standard at a concentration of 16 μg/mL in MeOH (100%), in negative mode. The standard shows a single peak with a retention time of 29.67 minutes and the pattern of fragmentation is shown on panel B (887.57-888.57-889.57-890.58 m/z). C) Quantification data of hpa are shown as percentage relative to LEGE 07175 (area of the peaks on the TIC on the selected mass range (887.50-888.50 m/z) with absorption at 428 nm on PDA).

### DETAILED DESCRIPTION

The present disclosure relates to hydroxypheophorbide compounds, in particular 13²-hydroxy-pheophorbide compounds, with bioactivities towards obesity and obesity-related co-morbidities.

Therefore, the present subject-matter discloses compounds or a pharmaceutically acceptable salt, hydrate, solvate, N-oxide, stereoisomer, diastereoisomer, enantiomer or atropisomer, polymorph for use in medicine comprising the following formula

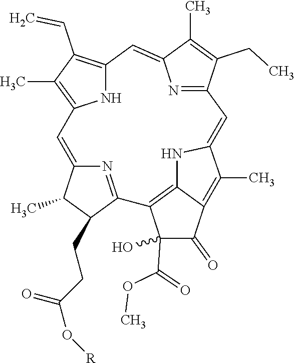

wherein R is CₙH₍₂ₙ₋₁₎ and n is an entire number multiple of 5. Preferably, n may be selected from 5 or 10 or 15 or 20 or 25 or 30, preferably 15 or 20.

The compounds of the present disclosure may be use in the therapy or treatment of obesity, an obesity-related disorder, an obesity related disease, being overweight, an obesity-related condition, or lipid obesity disorders.

### Structure of 13²(S)-hydroxy-pheofarnesin a (hfa)

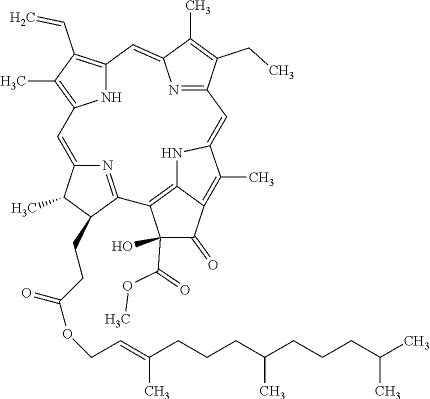 [SMILES: C=Cc1c(C)c2cc3nc(c4c5[nH]c(cc6nc(cc1[nH]2)C(C)=C6CC)c(C)c5C(=O)[C@]4(O)C(=O)OC)[C@@H](CCC(=O)OC/C=C(\C)CCCC(C)CCCC(C)C)[C@@H]3C]

### Structure of 13²(R)-hydroxy-pheofarnesin a

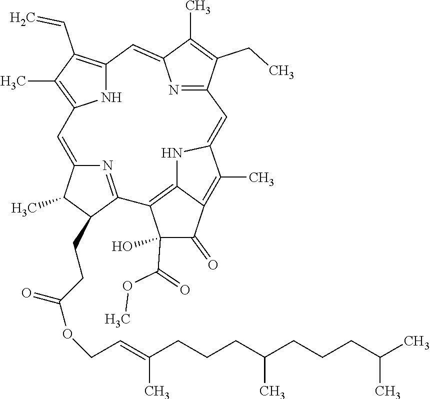 [SMILES: C=Cc1c(C)c2cc3nc(c4c5[nH]c(cc6nc(cc1[nH]2)C(C)=C6CC)c(C)c5C(=O)[C@@]4(O)C(=O)OC)[C@@H](CCC(=O)OC/C=C(\C)CCCC(C)CCCC(C)C)[C@@H]3C]

### Structure of 13²(S)-hydroxy-pheophytin a (hpa)

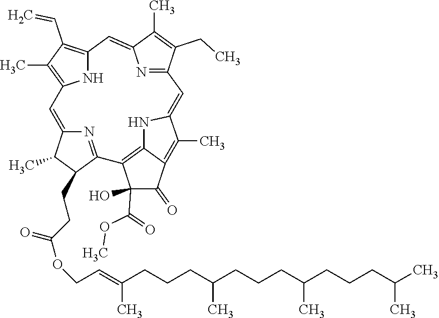 [SMILES: C=Cc1c(C)c2cc3nc(c4c5[nH]c(cc6nc(cc1[nH]2)C(C)=C6CC)c(C)c5C(=O)[C@]4(O)C(=O)OC)[C@@H](CCC(=O)OC/C=C(\C)CCCC(C)CCCC(C)CCCC(C)C)[C@@H]3C]

### Structure of 13²(R)-hydroxy-pheophytin a

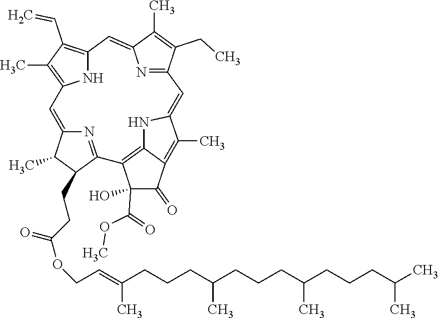 [SMILES: C=Cc1c(C)c2cc3nc(c4c5[nH]c(cc6nc(cc1[nH]2)C(C)=C6CC)c(C)c5C(=O)[C@@]4(O)C(=O)OC)[C@@H](CCC(=O)OC/C=C(\C)CCCC(C)CCCC(C)CCCC(C)C)[C@@H]3C]

This disclosure uncovers compounds with novel lipid reducing activity. It is reported the isolation procedure of two hydroxypheophorbide compounds (hpa and hfa), and the structural elucidation of hfa. Hpa was isolated from Cyanobium sp. LEGE07175 and hfa from Nodosilinea sp. LEGE06001. These marine cyanobacteria are commercially available and can be purchased at http://lege.ciimar.up.pt/ordering-services/). Their lipid reducing activity was evaluated using the zebrafish Nile red fat metabolism assay and a 3D cell culture model of murine pre-adipocytes. Furthermore, the production of hpa was evaluated in different materials (Spirulina, microalgae, spinach, cabbage), with GRAS status (generally regarded as safe) for human consumption, to foster the commercialization of the compounds as novel natural products in the form of nutraceuticals or food supplements for the treatment of obesity or related co-morbidities.

In an embodiment, cyanobacterial growth, extraction and fractionation were performed. Cyanobacteria strains Cyanobium sp. LEGE07175 and Nodosilinea sp. LEGE06001 were isolated from the Portuguese coast and are maintained in the LEGEcc in CIIMAR, Matosinhos, Portugal (the strains are commercial and can be purchased at http://lege.ciimar.up.pt/ordering-services/). The strains were cultured in Z8 medium supplemented with marine tropical salt (25 g/L), at 25° C., with a photoperiod of 14 h/10 h light and dark respectively, and at light intensity of 10 μmols photons s1m2. Nodosilinea sp. LEGE 06001 and Cyanobium sp. LEGE 07175 cultures were grown in 20 L flasks with constant aeration and at the exponential phase, cells were harvested through centrifugation, frozen and freeze-dried. The biomass of LEGE 07175 (13 g) and LEGE 06001 (56.5 g) was extracted by repeated percolation with warm mixture of CH2Cl2/MeOH (2:1, v/v) yielding a crude extract of 1.9 g and 8.74 g, respectively. Both crude extracts were fractionated by normal-phase (Si gel 60, 0.015-0.040 mm, Merck KGaA) VLC with an increasing polarity grade, from 90% n-hex to 100% EtOAc and 100% MeOH, giving a total of 9 fractions each.

In an embodiment, hpa was obtained as a green dark amorphous solid, after several chromatographic steps of an extract from the cyanobacterium Cyanobium sp. LEGE 07175 (the strain is commercially available at http://lege.ciimar.up.pt/ordering-services/). The isolation was guided by a strong the reduction of lipid content observed in the zebrafish Nile red fat metabolism assay. The structural assignment of hpa was obtained through comparison of the obtained spectroscopic data with those reported on the literature. The HR-ESI-MS analysis showed a monoisotopic m/z 887.5697 [M+H]⁺, consistent with the molecular formula of C₅₅H₇₄N₄O₆. The 1D and 2D NMR experiments analysis revealed the typical resonances and correlations for this compound. The presence of the alcohol group and other functional groups were corroborated using NMR and HR-ESI-MS/MS.

The bioassay guided fractionation of the extract of Nodosilinea sp LEGE 06001 (strain commercially available at http://lege.ciimar.up.pt/ordering-services/) yielded hfa as a brownish green amorphous solid. The molecular formula was assigned as C₅₀H₆₄N₄O₆ on the basis of HR-ESI-MS data, m/z 817.4505 [M+H]⁺, which demanded fifteen degrees of unsaturation.

The ¹H and ¹³C NMR data of hfa showed similarities to what was observed for hpa, indicating hfa to be a hydroxypheophorbide compound. Through the comparison of the HR-ESI-MS data it could be concluded that compound hfa bears a farnesyl moiety instead of a phytyl. The difference between the mass of the pseudomolecular ion at m/z 817.4505 [M+H]⁺ and the fragment at m/z 609.2702 [M—farnesyl]⁺ (Δm/z 208.1803) is consistent with the presence of a farnesyl substituent in opposition to phytyl (Δm/z 279.36; m/z 909.55 [M+Na]⁺ and m/z 607.20 [M—phytol]⁺. Therefore, compound hfa was named hydroxyl-pheofarnesine a.

Hence, the H NMR spectrum, showed all the typical resonances of the porphyrin ring: three singlets and one multiplet aromatic methyl groups (δ_{H} 1.59 m, 3.28 s, 3.44 s, 3.90 s), one aromatic ethyl group (δ_{H} 1.72, 3.64, 3.77), three olefinic singlets (δ 8.71, 9.56, 9.77), a methoxyl group (δ 3.76) and a vinyl substitution (δ_{H} 6.19, 6.35, 8.03) with a characteristic exomethylene coupling pattern (J=18.7 and J=11.4 Hz). The attached farnesyl moiety was recognized by the carbinol resonances (δ_{H} 4.44, 4.5 at F1), the vinylic resonances for H—F2 (δ_{H} 5.14) and H₃—F3¹ (δ_{H} 1.61), and the germinal methyl's at F11¹ and F12 (δ_{H} 0.85 d).

Zebrafish (Danio rerio) has become an attractive model organism for biomedical research. In the study of complex metabolic disorders like obesity, the use of more complex in vivo model systems as small whole animal models can bring significant advantages. For drug discovery, zebrafish assays can be used complementary to rodent assays, with easier handling, high predictive validity and cost-efficiency while compatible with high-throughput screening. Zebrafish possess higher physiological relevance than cellular in vitro models and have genetic homology to mammals, including significant similarities in lipid metabolism. The zebrafish Nile red fat metabolism assay analyses the capacity of compounds to reduce neutral lipids in zebrafish larvae in vivo.

13²(S)-hydroxy-pheophytin a (hpa) is a known compound, and belongs to the photosystem II complex of the chloroplast. The second is a novel compound, 13²(S)-hydroxy-pheofarnesin a (hfa), which is structurally related to hpa. Both compounds (hpa, hfa) have a lipid reducing activity in the zebrafish Nile red fat metabolism assay (Urbatzka et al., 2018) with IC₅₀ values of 8.9±0.4 and 15.5±1.3 μM, respectively. No general toxicity was observed for both compounds in the same concentration ranges on zebrafish larvae (death, malformations). In contrast, exposure of zebrafish larvae to chlorophyll a and chlorophyll b did not demonstrate any lipid reducing activity.

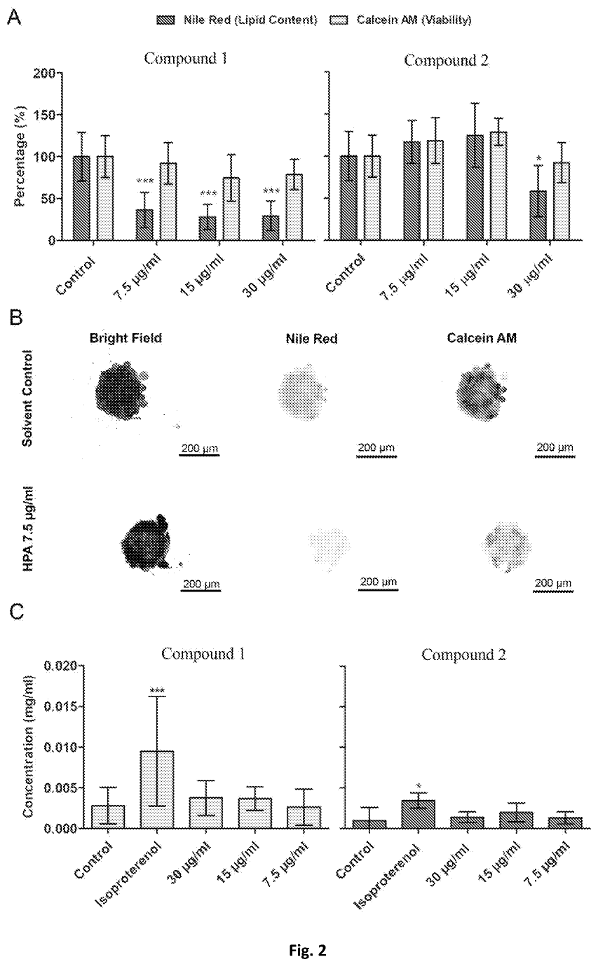

In an embodiment, the bioactivity of hpa and hfa was confirmed in a further assay, using the murine pre-adipocyte cell line 3T3L1. Cells grown and differentiated as spheroids were exposed to hpa and hfa, respectively, and reduced significantly the lipid content of the spheroids. No toxicity was observed by calcein AM staining, in contrast non-specific cellular esterase activity was increased. Lipolysis was analyzed as potential mechanism, but hpa or hfa exposure of spheroids did not alter lipolytic activities. In an embodiment, the activity in differentiated 3T3L1 spheroids was confirmed and analysis of lipolysis performed. 3T3-L1 spheroids exposed after 7 d differentiation, illustrated in FIG. 2, showed significant reduction of lipid accumulation after 48 h of exposure to hpa at 7.5, 15 and 30 μg/mL, and to hfa at 30 μg/mL. Exposure to 15 and 30 μg/mL of hpa reduced the viability of spheroids for 20%, although not significant, while exposure to hfa did not cause any reduction in viability at any concentration.

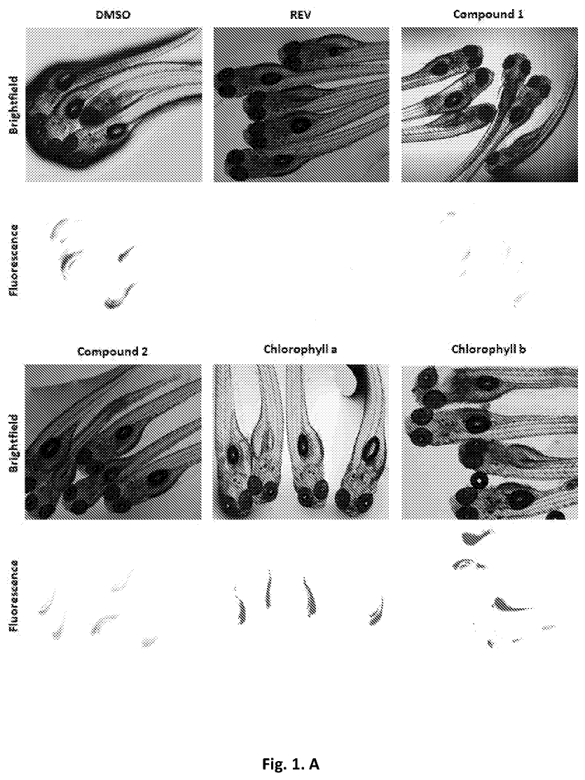

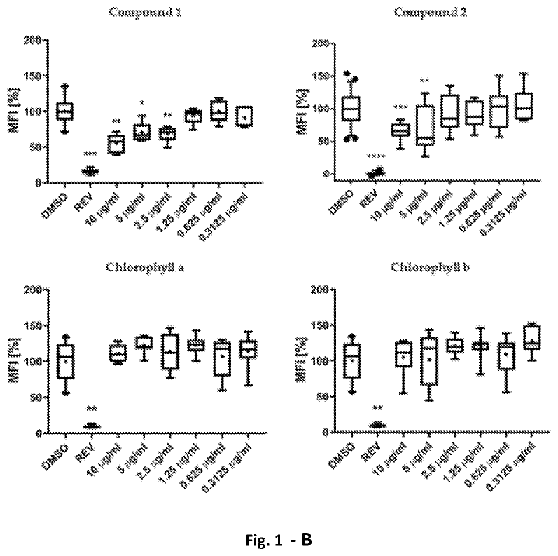

In an embodiment, chlorophyll a and chlorophyll b were tested for lipid reducing activity on the zebrafish Nile red fat metabolism assay. These compounds did not reduce the fluorescence intensity of Nile red in the assay at all tested concentrations (156 ng/mL to 10 μg/mL) as demonstrated in FIG. 1(B), highlighting the specificity of the disclosed hydroxypheophorbide compounds for lipid-reducing activity.

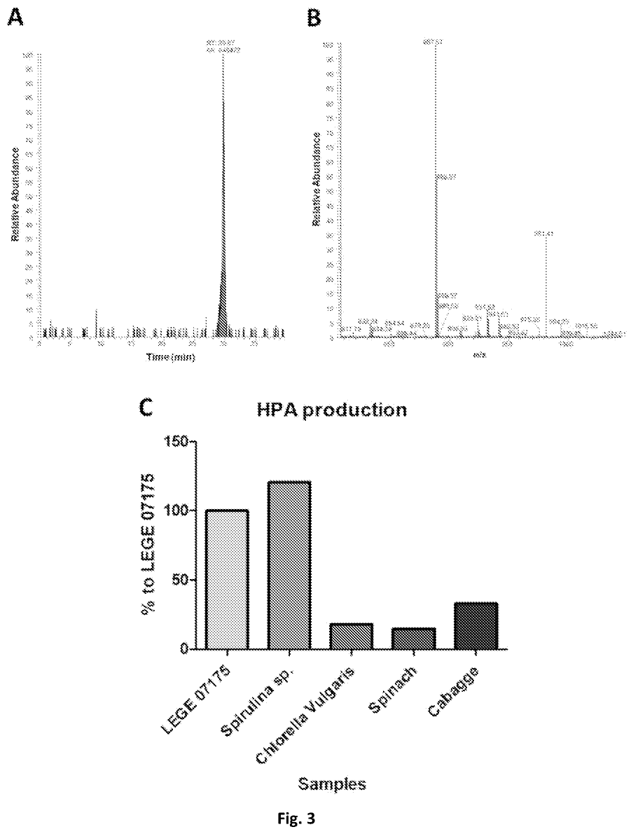

In an embodiment, to understand whether hpa could be found in biomass that is suitable for human consumption, the presence of this compound was analysed by LC-ESI-MS in in cyanobacteria from the LEGE cc collection, in Spirulina and Chlorella tabs from commercial sources, and in spinach and cabbage. When compared to Cyanobium sp. LEGE 07175 (100%), Spirulina sp. produced a higher amount of hpa (120.4%), while much lower values were observed for Chlorella vulgaris (18.0%), spinach (14.7%) and cabbage (33.0%), as shown in FIG. 3.

Synthesis of hpa, hfa, and other 13²-hydroxy-pheophorbides can be synthesized through the methanolic allomerization of chlorophyll a (Kuronen et al., 1993).

The synthesis of hfa can follow the route presented in scheme 1. Briefly, the acid hydrolysis of chlorophyll a will yield pheophorbide a (scheme 1, i) (You et al., 2011). The conversion of the carboxylic group of pheophorbide an into an acyl chloride can be done through its reaction with 3,3-dichlorocyclopropene (in basic conditions), or with thionyl chloride (scheme 1, ii). The subsequent esterification reaction of the acyl chloride intermediate with 3,7,11-trimethyl-2-dodecene-1-ol (scheme 1, iii), followed by methanolic allomerization will then yield hfa (scheme 1, iv). The same synthetic route can be adapted for the synthesis of other ester derivatives through the reaction of the acyl chloride intermediate with the respective alchools (scheme 1).

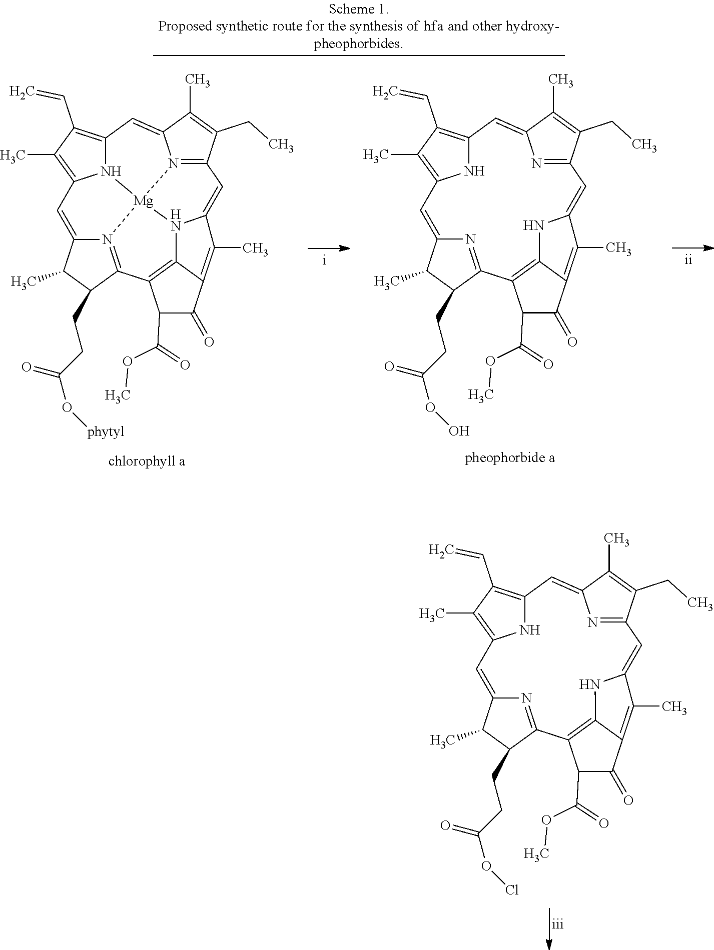

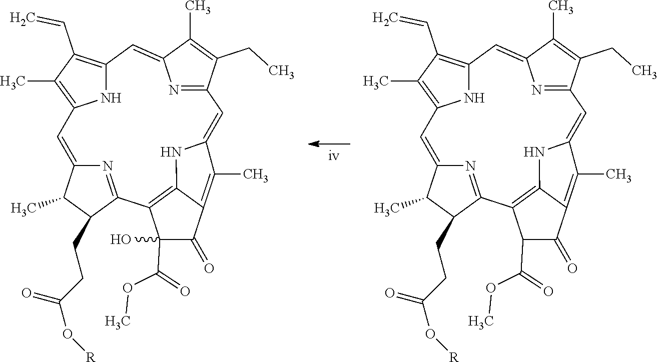

Since chlorophyll derivatives are common to all organisms with chloroplasts (plants, algae, microalgae), it can be expected that organism with a history of food consumption produce these compounds. Those histories of food consumption may lead to the selection of an organism that has the GRAS status (generally regarded as safe). The quantification of hpa in extracts of a LEGEcc cyanobacterial strain, Spirulina from a commercial source, Chlorella from a commercial source and from spinach and cabbage showed that hpa can be produced in large quantities in Spirulina, which has the GRAS status. Hydroxypheophorbide compounds may be developed as food supplements or nutraceuticals, or as an extract of microalgae rich in those compounds or even as dry biomass with a high content of the compounds—similar to Spirulina as nutraceutical with weight reducing properties. The application as concentrated pure compounds in the form of tablets, capsules or candies would be possible as well as the use of the whole dry mass of a plant/algae/microalgae rich in chlorophyll derivatives.

Where singular forms of elements or features are used in the specification of the claims, the plural form is also included, and vice versa, if not specifically excluded. For example, the term “a compound” or “the compound”” also includes the plural forms “compounds” or “the compounds,” and vice versa. In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

The term “comprising” whenever used in this document is intended to indicate the presence of stated features, integers, steps, components, but not to preclude the presence or addition of one or more other features, integers, steps, components or groups thereof.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim.

Additionally, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

The disclosure should not be seen in any way restricted to the embodiments described and a person with ordinary skill in the art will foresee many possibilities to modifications thereof.

The above described embodiments are combinable.

The following claims further set out particular embodiments of the disclosure.

All references recited in this document are incorporated herein in their entirety by reference, as if each and every reference had been incorporated by reference individually.
Castro, M.; Preto, M.; Vasconcelos, V.; Urbatzka, R. Obesity: The metabolic disease, advances on drug discovery and natural product research. Curr Top Med Chem 2016, 16, 2577-2604, doi: 10.2174/1568026616666160415155644.
Cheng, H. H.; Wang, H. K.; Ito, J.; Bastow, K. F.; Tachibana, Y.; Nakanishi, Y.; Xu, Z.; Luo, T. Y.; Lee, K. H. Cytotoxic pheophorbide-related compounds from Clerodendrum calamitosum and C. cyrtophyllum. J Nat Prod 2001, 64, 915-919, doi: 10.1021/np000595b.
D. G. Semaan, J. O. Igoli, L. Young, A. I. Gray, E. G. Rowan, E. Marrero, In vitro anti-diabetic effect of flavonoids and pheophytins from Allophylus cominia Sw. on the glucose uptake assays by HepG2, L6, 3T3-L1 and fat accumulation in 3T3-L1 adipocytes, J Ethnopharmacol, 216, 2018, 8-17, doi.org/10.1016/j.jep.2018.01.014.
Ferruzzi, M.; Böhm, V.; Courtney, P.; Schwartz, S. Antioxidant and antimutagenic activity of dietary chlorophyll derivatives determined by radical scavenging and bacterial reverse mutagenesis assays. J Food Sci Technol 2002, 67, 2589-2595, doi: 10.1111/j.1365-2621.2002.tb08782.x.
Gomes, R. A.; Teles, Y. C. F.; Pereira, Fd. O.; Rodrigues, L. Ad. S.; Lima, Ed. O.; Agra Md. F.; Souza, Md. F. Vd. Phytoconstituents from Sidastrum micranthum (A. St.-Hil.) Fryxell (Malvaceae) and antimicrobial activity of pheophytin a. Braz J Pharm Sci 2015, 51, 861-867, doi: 10.1590/51984-82502015000400012;
Hayashi, K.-l.; Nozaki, H.; Kamei, Y. Pheophytin A, a low molecular weight compound found in the marine brown alga Sargassum fulvellum, promotes the differentiation of PC12 cells. Int. J. Dev. Neurosci. 2007, 25, 63-68, doi: 10.1016/j.ijdevneu.2006.09.323
Kang, Y-R.; Park, J.; Jung, S. K.; Chang, Y. H. Synthesis, characterization, and functional properties of chlorophylls, pheophytins, and Zn-pheophytins. Food Chem 2018, 245, 943-950, doi: doi.org/10.1016/j.foodchem.2017.11.079.
Kuronen, P., Hyvarinen, K., Hynninen, P. H., & Kilpelainen, I. (1993). High-performance liquid chromatographic separation and isolation of the methanolic allomerization products of chlorophyll a. J Chromatogr A, 654(1), 93-104. doi:10.1016/0021-9673(93)83068-4.
Ramos, V.; Morais, J.; Castelo-Branco, R.; Pinheiro, A.; Martins, J.; Regueiras, A.; Pereira, A. L.; Lopes, V. R.; Frazão, B.; Gomes, D.; Moreira, C.; Costa, M. S.; Brûle, S.; Faustino, S.; Martins, R.; Saker, M.; Osswald, J.; Leão, P. N.; Vasconcelos, V. M. Cyanobacterial diversity held in microbial biological resource centers as a biotechnological asset: the case study of the newly established LEGE culture collection. J Appl Phycol 2018, 30, 1437-1451, doi: 10.1007/s10811-017-1369-y.
Subramoniam, A.; Asha, V. V.; Nair, S. A.; Sasidharan, S. P.; Sureshkumar, P. K.; Rajendran, K. N.; Karunagaran, D.; Ramalingam, K. Chlorophyll revisited: Anti-inflammatory activities of chlorophyll a and inhibition of expression of TNF-α gene by the same. Inflammation 2012, 35, 959-966, doi: 10.1007/s10753-011-9399-0.
Shu, M. H.; Appleton, D.; Zandi, K.; AbuBakar, S. Anti-inflammatory, gastroprotective and antiulcerogenic effects of red algae Gracilaria changii (Gracilariales, Rhodophyta) extract. BMC Complement Altern Med 2013, 13, 61, doi: 10.1186/1472-6882-13-61
Turner, P. A.; Harris, L. M.; Purser, C. A.; Baker, R. C.; Janorkar, A. V. A surface-tethered spheroid model for functional evaluation of 3T3-L1 adipocytes. Biotechnol Bioeng 2014, 111, 174-183, doi: 10.1002/bit.25099.
Urbatzka, R.; Freitas, S.; Palmeira, A.; Almeida, T.; Moreira, J.; Azevedo, C.; Afonso, C.; Correia-da-Silva, M.; Sousa, E.; Pinto, M.; Vasconcelos, V. Lipid reducing activity and toxicity profiles of a library of polyphenol derivatives. Eur J Med Chem 2018, 151, 272-284, doi: 10.1016/j.ejmech.2018.03.036.
Wang, S-Y.; Tseng, C-P.; Tsai, K-C.; Lin, C-F.; Wen, C-Y.; Tsay, H-S.; Sakamoto, N.; Tseng, C-H.; Cheng, J-C. Bioactivity-guided screening identifies pheophytin a as a potent anti-hepatitis C virus compound from Lonicera hypoglauca Miq. Biochem Biophys Res Commun 2009, 385, 230-235, doi: 10.1016/j.bbrc.2009.05.043
You, H., Yoon, H-E., Yoon, J-H., Ko, H., & Kim, Y-C. (2011). Synthesis of pheophorbide-a conjugates with anticancer drugs as potential cancer diagnostic and therapeutic agents. BioorgMed Chem, 19(18), 5383-5391. https://doi.org/10.1016/j.bmc.2011.07.058.
Zhao, Y.; Wang, X.; Wang, H.; Liu, T.; Xin, Z. Two new noroleanane-type triterpene saponins from the methanol extract of Salicornia herbacea. Food Chem 2014, 151, 101-109, doi: 10.1016/j.foodchem.2013.11.030.

1. A compound or a pharmaceutically acceptable salt, hydrate, solvate, N-oxide, stereoisomer, diastereoisomer, enantiomer, atropisomer, or polymorph thereof, comprising the following formula: 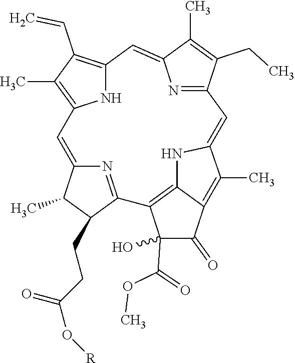
wherein R is CₙH₍₂ₙ₋₁₎ and n is an entire number multiple of 5.

2. The compound of claim 1, wherein n is 5, 10, 15, 20, 25, or 30.

3. The compound of claim 1, wherein R is C₅H₉, C₁₀H₁₉, C₁₅H₂₉, C₂₀H₃₉, C₂₅H₄₉, or C₃₀H₅₉.

4. The compound of claim 1, wherein R is

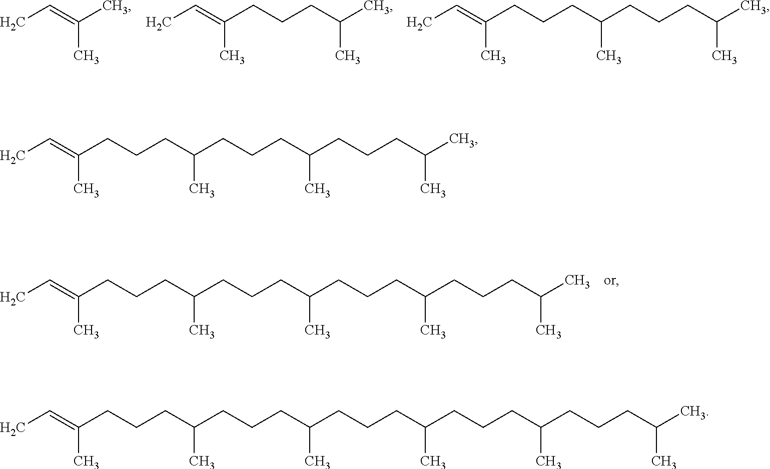

5. The compound of claim 1, wherein the formula is:

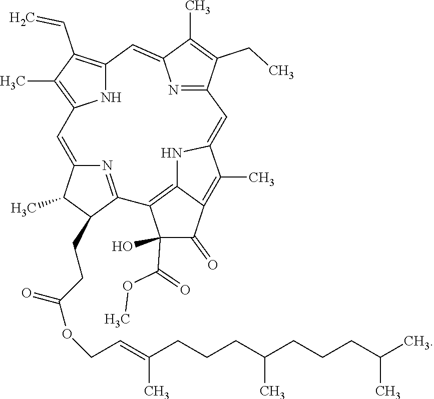 [SMILES: C=Cc1c(C)c2cc3nc(c4c5[nH]c(cc6nc(cc1[nH]2)C(C)=C6CC)c(C)c5C(=O)[C@]4(O)C(=O)OC)[C@@H](CCC(=O)OC/C=C(\C)CCCC(C)CCCC(C)C)[C@@H]3C]

6. The compound of claim 1, wherein the formula is:

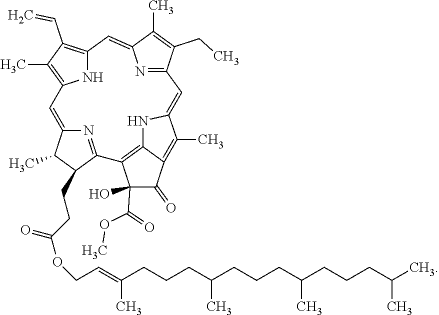 [SMILES: C=Cc1c(C)c2cc3nc(c4c5[nH]c(cc6nc(cc1[nH]2)C(C)=C6CC)c(C)c5C(=O)[C@]4(O)C(=O)OC)[C@@H](CCC(=O)OC/C=C(\C)CCCC(C)CCCC(C)CCCC(C)C)[C@@H]3C]

7. The compound of claim 1, wherein the compound is suitable for therapy or treatment of metabolic diseases.

8. The compound of claim 1, wherein the compound is suitable for therapy or treatment of obesity, an obesity-related disorder, an obesity related disease, being overweight, an obesity-related condition, or lipid obesity disorders.

9. The compound of claim 1, wherein the compound is suitable for therapy or treatment of morbid obesity, visceral fat obesity, diabetes, insulin-resistance syndrome, nonalcoholic steatohepatitis, a cardiovascular disorder, renal disorders, pancreatic disorders, polycystic ovary syndrome, or metabolic syndrome or fatty liver.

10. The compound of claim 1,
wherein
n is different from 20.

11. The compound of claim 10, wherein n is 15.

12. The compound of claim 10, wherein R is C₅H₉, C₁₀H₁₉, C₁₅H₂₉, C₂₅H₄₉, or C₃₀H₅₉.

13. The compound of claim 10, wherein R is

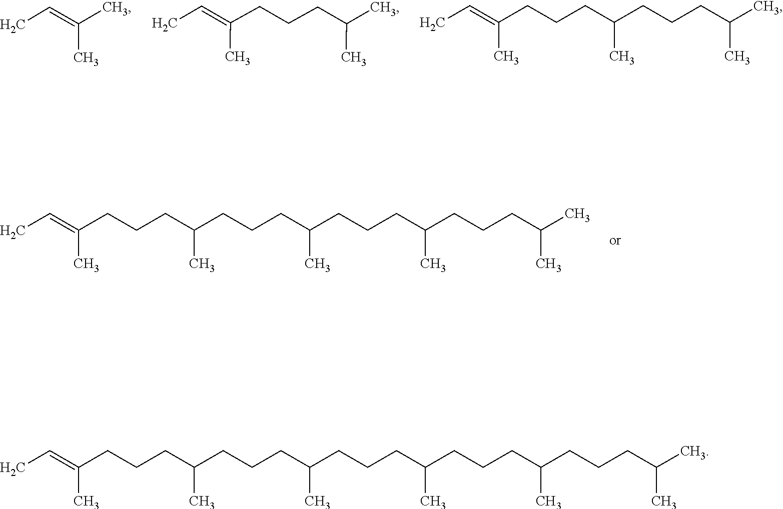

14. (canceled)

15. A composition comprising the compound of claim 1.

16. The composition of claim 15, wherein the composition is a pharmaceutical composition, a foodstuff composition, a dietary composition, a nutraceutical composition or an anti-obesity composition.

17. (canceled)

18. (canceled)

19. (canceled)

20. A method of treating or preventing obesity or related diseases comprising administering a therapeutically effective amount of the compound of claim 1.

21. A process for the production of the compound of claim 1, wherein the compound comprises the formula 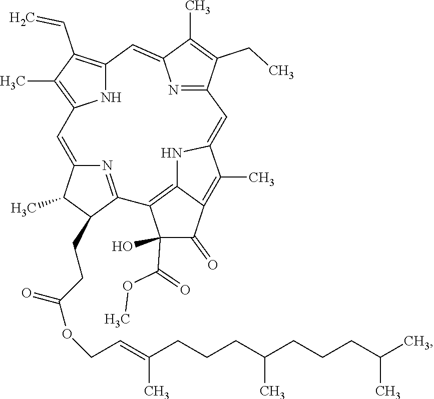 [SMILES: C=Cc1c(C)c2cc3nc(c4c5[nH]c(cc6nc(cc1[nH]2)C(C)=C6CC)c(C)c5C(=O)[C@]4(O)C(=O)OC)[C@@H](CCC(=O)OC/C=C(\C)CCCC(C)CCCC(C)C)[C@@H]3C]
and is isolated from an extract of Nodosilinea sp. LEGE06001.

22. A process for the production of the compound of claim 1, wherein the compound comprises the formula 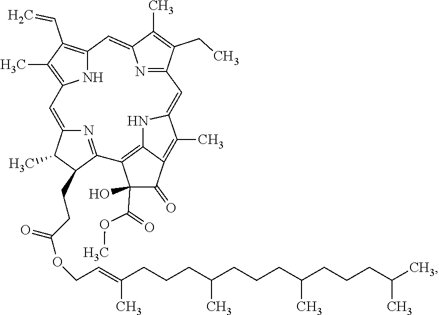 [SMILES: C=Cc1c(C)c2cc3nc(c4c5[nH]c(cc6nc(cc1[nH]2)C(C)=C6CC)c(C)c5C(=O)[C@]4(O)C(=O)OC)[C@@H](CCC(=O)OC/C=C(\C)CCCC(C)CCCC(C)CCCC(C)C)[C@@H]3C]
and is isolated from an extract of Cyanobium sp. LEGE 07175.
